(19) United States
(12) Patent Application Publication
(10) Pub. No.: US 2014/0134686 A1
(43) Pub. Date: May 15, 2014

(54) BIOMASS LIQUEFACTION THROUGH GAS FERMENTATION

(71) Applicant: LanzaTech New Zealand Limited, Auckland (NZ)

(72) Inventors: Michael Anthony Schultz, Roselle, IL (US); Jennifer Rosa Holmgren, Roselle, IL (US)

(21) Appl. No.: 14/076,916

(22) Filed: Nov. 11, 2013

Related U.S. Application Data
(60) Provisional application No. 61/725,349, filed on Nov. 12, 2012.

(51) Int. Cl. C12P 7/54; C12P 7/06

(52) U.S. Cl. CPC C12P 7/54; C12P 7/065
USPC 435/140; 435/161; 435/158

(57) ABSTRACT

The invention provides methods and systems for the production of at least one product from the microbial fermentation of a gaseous susbtrate, wherein the gaseous substrate is derived from a biomass liquefaction process. The invention provides a method for improving efficiency of the fermentation by passing biomass accumulated in the fermentation process to the biomass liquefaction process for conversion to a gaseous substrate. In a particular aspect of the invention, the biomass liquefaction process is selected from pyrolysis or torrefaction.

### FIELD

This invention relates generally to methods for producing products, particularly alcohols, by microbial fermentation. In particular, the invention relates to methods for producing fermentation products from industrial gases associated with biomass liquefaction. The invention provides a method for producing at least one fermentation product by microbial fermentation of a gaseous substrate produced by a biomass liquefaction process such as torrefaction or pyrolysis.

### BACKGROUND

Catalytic processes may be used to convert gases consisting primarily of CO and/or CO and hydrogen (H₂) into a variety of fuels and chemicals. Micro-organisms may also be used to convert these gases into fuels and chemicals. These biological processes, although generally slower than chemical reactions, have several advantages over catalytic processes, including higher specificity, higher yields, lower energy costs and greater resistance to poisoning.

The ability of micro-organisms to grow on CO as a sole carbon source was first discovered in 1903. This was later determined to be a property of organisms that use the acetyl coenzyme A (acetyl CoA) biochemical pathway of autotrophic growth (also known as the Woods-Ljungdahl pathway and the carbon monoxide dehydrogenase/acetyl CoA synthase (CODH/ACS) pathway). A large number of anaerobic organisms including carboxydotrophic, photosynthetic, methanogenic and acetogenic organisms have been shown to metabolize CO to various end products, namely CO₂, H₂, methane, n-butanol, acetate and ethanol. In addition to these products, the inventors have previously demonstrated that a number of other useful products carbon-based products may be obtained by fermentation using specific microorganisms or those that express particular genes.

Anaerobic bacteria, such as those from the genus Clostridium, have been demonstrated to produce ethanol from CO, CO₂ and H₂ via the acetyl CoA biochemical pathway. For example, various strains of Clostridium ljungdahlii that produce ethanol from gases are described in WO 00/68407, EP 117309, U.S. Pat. Nos. 5,173,429, 5,593,886, and 6,368,819, WO 98/00558 and WO 02/08438. The bacterium Clostridium autoethanogenum sp is also known to produce ethanol from gases (Abrini et al., Archives of Microbiology 161, pp 345-351 (1994)).

Although processes for the fermentation of substrates containing CO and H₂ by microorganisms are known, the potential for scaling and integrating these processes into an industrial context has barely been explored. Petrochemical plants and oil refineries produce large quantities of CO as “waste” by-products. A significant proportion of the waste gases are currently sent to flare (burned), or alternatively used as a source of fuel, both of which produce the undesirable greenhouse gas CO₂. Accordingly, there exists the potential to make improvements to industrial processes by exploiting the waste gases and energy produced thereby for use in fermentation to produce desirable products while simultaneously reducing gaseous carbon emissions from industrial plants.

Liquefaction of biomass can be an economical way to obtain valuable liquid products. Biomass can be any type of woody biomass, agricultural waste, pulp and paper waste, municipal solid waste, or coal/coke. Three key processes for biomass conversion are torrefaction, pyrolysis and gasification.

Torrefaction involves subjecting biomass to relatively low temperatures (150-300 deg. C.) in the absence of air or oxygen. Volatile materials are created and then driven off, producing a densified carbon rich solid similar to coal. The gas stream produced contains CO and CO₂.

Pyrolysis is a thermochemical decomposition of organic material at elevated temperatures (typically at temperature above 450-500 deg. C.) without the participation of oxygen. It involves the simultaneous change of chemical composition and physical phase, and is irreversible. Pyrolysis can be characterized as ‘fast’ or ‘slow’, or somewhat in between, describing the relative time under reaction conditions. Gas, liquid, and solid products are produced, with the relative amounts depending on the temperature and reaction time. Fast pyrolysis maximizes liquid yield but is more challenging.

Gasification of biomass involves the use of oxygen/air/steam to produce syngas.

It is an object of the invention to provide an integrated method and/or system comprising biomass liquefaction and gas fermentation to produce useful products, or at least to provide the public with a useful choice.

### SUMMARY OF INVENTION

The invention generally provides, inter alia, methods for the production of products by microbial fermentation of a substrate comprising CO.

In a first aspect, the invention provides a method of production of at least one product, the method including:
i) providing a substrate comprising CO to a bioreactor containing a culture of at least one carboxydotrophic acetogenic micro-organism; and
ii) fermenting the culture in the bioreactor to produce the at least one fermentation product,
   wherein the substrate of step (i) is derived from one or more biomass liquefaction processes.

In a particular embodiment, the substrate comprising CO further comprises CO₂ and/or H₂.

In a particular embodiment, the substrate comprising CO is synthesis gas.

In a particular embodiment the syngas is separated to provide a CO comprising substrate and a CO2/H2 gaseous stream. In one embodiment the CO comprising substrate is passed to a first bioreactor where it is fermented to produce one or more alcohols and/or acids and an exit gas stream comprising CO2. In certain embodiments the exit gas stream further comprises hydrogen. In particular embodiments, the exit gas stream is combined with the CO2/H2 gaseous stream. In certain embodiments the combined stream is passed to a second bioreactor containing a culture of one or more micro-organisms, and fermented to produce acetate.

In a particular embodiment, the one or more biomass liquefaction process is selected from torrefaction, pyrolysis and gasification. In one embodiment the biomass liquefaction process is carried out in a liquefaction zone.

In a particular embodiment, the biomass liquefaction process is adapted so as to produce a liquefaction gas product particularly suitable for use in a gas fermentation process. For example increasing the temperature of the liquefaction process produces carbon monoxide preferentially to carbon dioxide. Fuet et al., discuss variations between gas, liquid and char production at varying temperatures (Fu et. Al., Bioresource Technology, Vol. 102, Issue 17, September 2011, Pg 8211-8219). In a particular embodiment, the liquefaction gas product comprises CO at a concentration of from between about 20% to about 60%. In a particular embodiment, the liquefaction gas product comprises CO₂ at a concentration of from 0% to about 40%. In a particular embodiment, the liquefaction gas product comprises CO₂ at a concentration of from 0% to 10%. In a particular embodiment, the liquefaction gas product comprises a mixture of gases comprising CO at a concentration as described above, in combination with CO₂ at a concentration as described above and/or H₂ at a concentration as described above.

In a further particular embodiment, the liquefaction gas product comprises impurities selected from the group consisting of NH₃, NO, H₂S, HCN, SO₂ and SO₃. In a particular embodiment, at least a portion of the biomass used in the biomass liquefaction process comprises biomass recovered from the bioreactor.

In a particular embodiment, the energy produced during the liquefaction process may be used to increase the efficiency of the fermentation reaction and subsequent separation of fermentation products. In particualr emboidments, the energy is used to heat or cool the fermentation substrate, or to enable separation of fermentation products, for example by distillation.

In a particular embodiment, the biomass liquefaction process comprises pyrolysis and a pyrolysis product is produced. Preferably, the pyrolysis product is pyrolysis oil, char and/or pyrolysis gas.

In a particular embodiment, at least a portion of the pyrolysis gas is passed to the bioreactor as part of the substrate comprising CO.

In a particular embodiment, the pyrolysis oil is contacted with an outlet gas stream comprising hydrogen received from the bioreactor. When the substrate comprising CO provided to the bioreactor also comprises H₂, the fermentation process fixes the CO and optionally CO₂ components of the substrate thus resulting in the outlet gas stream having a higher concentration of hydrogen. The fermentation effectively acts as a hydrogen membrane allowing H₂ to pass through unconverted and to concentrate the H₂ in the outlet gas stream compared to the substrate provided to the bioreactor. Preferably, the outlet gas stream comprising hydrogen contacts the pyrolysis oil and hydrogenates the oil to produce a hydrocarbon product having from 6 to 20 carbons. In one embodiment the hydrocarbon product is high grade kerosene suitable for use as jet fuel (JP-5, JP-8) or other processes requiring high purity kerosene. In certain embodiments, the outlet stream from the upgrading process can be used up stream as a fuel source.

In a particular embodiment, a solid and/or liquid pyrolysis product undergoes gasification and at least a portion of the gasified product is passed to the bioreactor as part of the substrate comprising CO. Preferably, the solid pyrolysis product is char and the liquid pyrolysis product is pyrolysis oil.

In a particular embodiment, the char undergoes conversion in the presence of CO₂ to form CO for addition to the substrate comprising CO. Preferably, the CO₂ for conversion is received from the bioreactor, a torrefaction process and/or a pyrolysis process.

In a particular embodiment, the biomass liquefaction process comprises torrefaction. In a particular embodiment, the torrefaction process produces one or more torrefaction gases comprising CO, CO₂ and/or H₂. Preferably, at least a portion of the one or more torrefaction gases is added to the substrate comprising CO to be passed to the fermentation process.

In a particular embodiment, the one or more biomass liquefaction processes comprises torrefaction and pyrolysis. Preferably, biomass is first subjected to torrefaction then at least a portion of at least one torrefaction product is subjected to pyrolysis after which at least a portion of at least one pyrolysis product is added to the substrate comprising CO for use in the gas fermentation.

In a particular embodiment, the substrate comprising CO further comprises CO₂ wherein the CO₂ is a product of a torrefaction, gasification or pyrolysis process.

In a particular embodiment, the substrate comprising CO further comprises H₂ wherein the H₂ is a product of a torrefaction, pyrolysis or gasification process.

In a particular embodiment, the one or more fermentation products are an alcohol or diol. In one embodiment the alcohol is ethanol. In an alternative embodiment the diol is 2,3-butanediol. In certain embodiments the one or more fermentation products is ethanol and 2,3-butanediol.

In a particular embodiment, the one or more fermentation products are converted to one or more alkanes.

In a particular embodiment, one or more arene compounds are obtained from the pyrolysis oil. In a further embodiment, the one or more arene compounds are combined with one or more alkanes to produce a fuel.

In a second aspect, the invention provides a method for producing at least one product from a gaseous substrate, the method comprising:
a. Passing a biomass feedstock to a pyrolysis zone, operated at conditions to produce a gaseous substrate comprising CO, CO₂ and H₂, and at least one pyrolysis product, selected from the group consisting of pyrolysis oil and char;
b. Passing at least a portion of the gaseous substrate to a bioreactor comprising a culture of at least one carboxydotrophic acetogenic microorganism, and anaerobically fermenting at least a portion of the gaseous substrate to produce at least one fermentation product, a waste stream comprising a second biomass and an exit gas stream comprising hydrogen;
c. Separating at least a portion of the second biomass from the waste stream; and
d. Passing a portion of the second biomass to the pyrolysis zone.

In one embodiment the exit gas stream is passed to a separation zone operated at conditions to provide a hydrogen rich stream. In one embodiment the pyrolysis product is pyrolysis oil, and the hydrogen rich stream and the pyrolysis oil are passed to hydrogenation zone operated at conditions to provide a hydrogenated product. In one embodiment the hydrogenated product is a hydrocarbon having between 6 and 20 carbons. In one embodiment the hydrogenation zone is operated at conditions to provide a jet fuel hydrocarbon product.

In one embodiment the gaseous substrate from the pyrolysis zone comprising CO, CO₂ and H₂, is passed to a separation zone operated at conditions to provide a CO₂ richstream and an enriched CO and H₂ gaseous substrate. In one embodiment the enriched CO and H₂ stream is passed to the bioreactor.

In one embodiment the pyrolysis product is char and the char and the CO₂ rich stream are passed to a reaction zone, operated at conditions to produce a second substrate stream comprising CO. In one embodiment, the second substrate stream comprising CO is passed to the bioreactor.

In one embodiment, the pyrolysis product is pyrolysisi oil and/or char, and the pyrolysis prodcut is passed to a gasification zone operated at conditions to produce a gasified substrate comprising CO.

In one embodiment, the biomass passed to the pyrolysis zone, is first passed to a pre-treatment zone. In one embodiment the pre treatment zone is a torrefaction zone. In one embodiment the biomass is passed to the torrefaction zone, operated at coniditons to produce a terrified biomass. The terrified biomass is then passed to the pyrolysis zone.

In a third aspect, the invention provides a method for producing at least one product from a gaseous substrate, the method comprising;
a. passing a biomass feedstock to a torrefaction zone to produce a torrefied biomass;
b. passing the torrefied biomass to a pyrolysis zone to produce a gaseous substrate comprising CO, pyrolysis oil and char;
c. Passing at least a portion of the gaseous substrate to a bioreactor comprising a culture of at least one carboxydotrophic acetogenic microorganism, and
anaerobically fermenting at least a portion of the gaseous substrate to produce at least one fermentation product, a waste stream comprising a second biomass and an exit gas stream comprising hydrogen;
d. separating at least a portion of the second biomass from the waste stream;
e. passing the exit gas stream to a separation zone operated at conditions to provide a hydrogen rich stream;
f. passing a portion of the second biomass to the torrefaction zone; and
g. passing the pyrolysis oil and the hydrogen rich stream to a hydrogenation zone operated at conditions to produce a hydrogenated product.

In one embodiment the torrefaction process produces a gaseous by-product stream comprising CO and at least a portion of the gaseous by-product stream is passed to the bioreactor.

In one embodiment the cahr is passed to a gasification zone and gasified to produce a second gaseous substrate comprising CO. In one embodiment the second gaseous substrate comprising CO is passed to the bioreactor.

In one embodiment at least two of the gas streams selected from the group consisting of the gaseous by-product stream, the second gaseous stream, or the gaseous substrate produced by the pyrolysis reaction are blended prior to being passed to the bioreactor.

In one embodiment, the hydrogenated product is a hydrocarbon product having from 6 to 20 carbons. In one embodiment the hydrogenated product is a high grade kerosene. In one embodiment the hydrogenation zone is operated at conditions to produce a jet fuel hydrocarbon product.

In a fourth aspect, the invention provides a system for the production of a fermentation product comprising:
a bioreactor containing a culture of one or more micro-organisms adapted to produce the fermentation product by fermentation of a substrate comprising CO,
wherein the bioreactor is adapted to receive at least a portion of the substrate comprising CO from one or more biomass liquefaction processes.

In a particular embodiment, the substrate comprising CO further comprises CO₂ and/or H₂.

In a particular embodiment, the one or more biomass liquefaction processes is selected from torrefaction, pyrolysis and gasification.

In a particular embodiment, at least a portion of the biomass used in the biomass liquefaction process comprises biomass recovered from the bioreactor.

In a particular embodiment, the biomass liquefaction process comprises pyrolysis and the system further comprises a pyrolysis zone adapted to produce a pyrolysis product. Preferably, the pyrolysis product is pyrolysis oil, char and/or pyrolysis gas.

In a particular embodiment, the pyrolysis zone comprises at least one outlet adapted to pass at least a portion of a pyrolysis gas to the bioreactor.

In a particular embodiment, system further comprises a hydrogenation zone adapted to receive:
a. pyrolysis oil from the pyrolysis reactor; and
b. an outlet gas stream comprising hydrogen from the bioreactor.

In certain embodiments, the outlet gas stream is returned to pyrolysis zone. In a particular embodiment, the outlet gas stream us used as a fuel source.

In a particular embodiment, the system further comprises a gasification zone adapted to receive solid and/or liquid biomass and adapted to pass a gasified product to the bioreactor as part of the substrate comprising CO. Preferably, the solid and/or liquid biomass is a pyrolysis product received from the pyrolysis zone. Preferably, the solid pyrolysis product is char and the liquid pyrolysis product is pyrolysis oil.

In a particular embodiment, the system further comprises a torrefaction zone adapted to subject biomass to torrefaction to produce a terrified biomass and, an outlet adapted to pass at least a portion of one or more torrefaction gases comprising CO, CO₂ and/or H₂ to the bioreactor.

In a particular embodiment, the pyrolysis zone is adapted to receive at least a portion the torrified biomass.

In a particular embodiment, the pyrolysis zone, the torrefaction zone and/or the gasification zone further comprise one or more outlets adapted to pass at least a portion of one or more gas products to the bioreactor. Preferbly, the gas product comprises CO, CO₂ and/or H₂.

In a particular embodiment, the system comprises a char conversion zone adapted to convert char in the presence of CO₂ to form CO for addition to the substrate comprising CO. Preferably, the CO₂ for conversion is received via a gas recycling conduit from the bioreactor, the torrefaction reactor, the gasification module and/or the pyrolysis reactor.

In a fifth aspect, the invention provides a fermentation product when produced by the method of any of the first, second or third aspect, or the system of the fourth aspect.

The embodiments to follow can apply to any of the aspect provided herein.

In one embodiment tha carboxydotrophic acetogenic microorganism is from the genus Clostridium. In one embodiment the carboxydotrophic acetogenic microorganism is selected from the group consisting of Clostridium autoethanogenum, Clostridium ljungdahlii, Clostridium ragsdalei and Clostridium coskatii. In one particular embodiment, the microorganism is Clostridium autoethanogenum DSM23693. In another particular embodiment, the microorganism is Clostridium ljungdahlii DSM13528

In one embodiment, the fermentation product is an alcohol or diol. In one embodiment the alcohol is ethanol. In an alternative embodiment the diol is 2,3-butanediol. In certain embodiments the one or more fermentation products is ethanol and 2,3-butanediol. In one embodiment acetic acid is produced as a by-product of the fermentation.

In one embodiment, the invention provides one or more alkanes obtained as a derivative of the one or more fermentation products. In one embodiment the one or more fermentation product is further converted to downstream products by known conversion methods, such as thermochemcical or catalytic conversion methods.

In one embodiment the invention provides one or more arene compounds obtained as a derivative of a pyrolysis oil produced according to the method of any of the above aspects. In a particular embodiment, the one or more arene compounds are combined with one or more alkanes to produce a fuel.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

### BRIEF DESCRIPTION OF THE FIGURES

These and other aspects of the present invention, which should be considered in all its novel aspects, will become apparent from the following description, which is given by way of example only, with reference to the accompanying figures, in which:

FIG. 1: Exemplary integrated scheme showing a system of the invention comprising biomass liquefaction processes

FIG. 2: Exemplary integrated scheme showing a system and method for production of one or moe products by fermentation of gaseous substrates derived from biomass liquefaction process, according to a second aspect of the invention

### DETAILED DESCRIPTION OF THE INVENTION

### Definitions

The following is a description of the present invention, including preferred embodiments thereof, given in general terms.

As referred to herein, a “fermentation broth” is a culture medium comprising at least a nutrient media and bacterial cells.

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation.

The phrase “substrate comprising carbon monoxide” and like terms should be understood to include any substrate in which carbon monoxide is available to one or more strains of bacteria for growth and/or fermentation, for example. The substrate may be a “gaseous substrate comprising carbon monoxide” and like phrases and terms include any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for the substrate to contain any hydrogen, the presence of H₂ should not be detrimental to product formation in accordance with methods of the invention. In particular embodiments, the presence of hydrogen results in an improved overall efficiency of alcohol production. For example, in particular embodiments, the substrate may comprise an approx 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

In the description which follows, embodiments of the invention are described in terms of delivering and fermenting a “gaseous substrate containing CO”. However, it should be appreciated that the gaseous substrate may be provided in alternative forms. For example, the gaseous substrate containing CO may be provided dissolved in a liquid. Essentially, a liquid is saturated with a carbon monoxide containing gas and then that liquid is added to the bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October, 2002) could be used. By way of further example, the gaseous substrate containing CO may be adsorbed onto a solid support. Such alternative methods are encompassed by use of the term “substrate comprising CO” and the like.

In particular embodiments of the invention, the CO-containing gaseous substrate is an industrial off or waste gas. “Industrial waste or off gases” should be taken broadly to include any gases comprising CO produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. In some embodiments the bioreactor may comprise a first growth reactor and a second or further fermentation reactor. As such, the addition of metals or compositions to a fermentation reaction should be understood to include addition to any one of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments the bioreactor may comprise a first growth reactor and a second or further fermentation reactor. As such, when referring to the addition of substrate to the bioreactor or fermentation reaction it should be understood to include addition to any one of these reactors where appropriate.

### DESCRIPTION

The inventors have surprisingly found that an integrated system comprising a biomass liquefaction process and a gas fermentation process may be used to produce useful fermentation products. The biomass liquefaction processes may be adapted to produce a gaseous substrate particularly suited for use in a gas fermentation process.

The CO and CO₂ and/or H₂ is captured or channelled from the biomass liquefaction process using any convenient method. Depending on the composition of the gaseous substrate, it may also be desirable to treat it to remove any undesired impurities, before introducing it to the fermentation. For example, the substrate may be filtered or scrubbed using known methods. However, the inventors have found that the microbial culture used in the fermentation has a surprisingly high tolerance to impurities that may be found in the liquefaction products. While the liquefaction gas products may be relatively impure and appear to be unsuitable for use in a microbial fermentation the fermentation is in fact able to proceed and produce useful fermentation products.

In addition, the ultimate and relative concentration of the gases CO, CO₂ and/or H₂ may be optimised for a microbial fermentation by adjusting particular liquefaction process parameters. For example by keeping temperatures lower during the liquefaction process, liquid yields can be maximised. However by using a hotter temperature during the liquefaction process, will result in increased amount of CO. This increase in CO can be beneficial to the fermentation process, as an increase in CO gas at this stage will enable an increased ethanol yield during ther fermentation process.

### Microbial Biomass

The invention also provides an integrated system for the recycling of biomass from fermentation. In this embodiment of the invention, at least a portion of the biomass used in the biomass liquefaction process comprises biomass recovered from the bioreactor. The recovered biomass consists predominantly of dead cellular matter from the microorganism culture.

The biomass is removed and recycled to be processed by one or more biomass liquefaction processes, such as those described herein. It may be desirable to treat the removed biomass prior to liquefaction to remove moisture, fermentation products or to modify its characteristics in other ways.

Known liquefaction processes often use biomass comprising agricultural waste and other common biomass sources. However, these feedstocks often contain particle sizes that are too high for optimal liquefaction processing. The present invention provides a biomass feedstock recovered from a microbial fermentation. This fermentation biomass has a small particle size and is straightforward to prepare as a dry, finely divided biomass feedstock appropriate for efficient liquefaction processing.

### Torrefaction

Torrefaction is a thermo-chemical treatment of biomass in the 150 to 340 degrees Celsius range in the absence of oxygen. In this process the biomass partly (especially the hemi-cellulose) decomposes, giving off various types of volatiles. The remaining torrefied biomass (solid) has approximately 30% more energy content per unit of mass. The torrefaction process produces CO, CO₂ and/or H₂ which can be used in the fermentation processes described herein. In a particular embodiment, at least a portion of one or more torrefaction gases is added to the substrate comprising CO to be passed to the fermentation process.

In a particular embodiment, the one or more biomass liquefaction processes comprises torrefaction and pyrolysis. Preferably, biomass is first subjected to torrefaction to produce torrified biomass, and then at least a portion of the torrified biomass is subjected to pyrolysis. At least one gaseous product of the pyrolysis process is passed to the bioreactor. In one embodiment, at least one gaseous product of the pyrolysis process is added to the substrate comprising CO produced by the torrefaction process. In one embodiment the at least one gaseous product of the pyrolysis process is selected from the group consisting of CO, CO₂ and H₂.

In a particular embodiment, the substrate comprising CO further comprises CO₂ wherein the CO₂ is a product of a torrefaction, gasification or pyrolysis process.

In a particular embodiment, the substrate comprising CO further comprises H₂ wherein the H₂ is a product of a pyrolysis, torrefaction or gasification process.

In a particular embodiment, the system further comprises a torrefaction zone adapted to subject biomass to torrefaction to produce a torrified biomass and, an outlet adapted to pass at least a portion of one or more torrefaction gases comprising CO, CO₂ and/or H₂ to the bioreactor.

In one embodiment the pyrolysis zone is adapted to receive at least a portion of the torrified biomass from the torrefaction zone.

In a particular embodiment the pyrolysis zone, the torrefaction zone and/or the gasification zone further comprise one or more outlets adapted to pass at least a portion of one or more gas products to the bioreactor. Preferbly, the gas product comprises CO, CO₂ and/or H2.

In one embodiment, the gaseous products produced by any one of the torrefaction zone, the pyrolysis zone, or the gasification zone is passed to a separation zone operated to separate at least one portion of the gas stream(s). In one embodiment the separation zone is operated under conditions to separate CO₂ from the gas stream to produce a CO₂ rich stream, and a CO and H₂ enriched stream.

### Pyrolysis

In a particular embodiment, the biomass liquefaction process comprises pyrolysis. The pyrolysis process prodcues a gaseous substrate comprising CO and a pyrolysis product. The pyrolysis product is selected from the group consisting of pyrolysis oil and char. Pyrolysis produces these products from biomass by heating the biomass in a low/no oxygen environment. The absence of oxygen prevents combustion. The relative yield of products from pyrolysis varies with temperature. Temperatures of 400-500° C. (752-932° F.) produce more char, while temperatures above 700° C. (1,292° F.) favor the yield of liquid and gas fuel components.

Pyrolysis occurs more quickly at the higher temperatures, typically requiring seconds instead of hours. High temperature pyrolysis is also known as gasification, and produces primarily syngas. Typical yields are 60% pyrolysis oil (also known as bio-oil), 20% biochar, and 20% syngas. By comparison, slow pyrolysis can produce substantially more char (˜50%). Once initialized, both processes produce net energy. For typical inputs, the energy required to run a “fast” pyrolyzer is approximately 15% of the energy that it outputs.

In a particular embodiment, the energy produced during the liquefaction process may be used to increase the efficiency of the fermetnation reaction and subsequent separation of fermentation products. In particular embodiments, the energy is used to heat or cool the fermentation substrate, or to enable separation of fermentation products, for example by distillation.

“Fast” pyrolysis has the advantage that it operates at atmospheric pressure and modest temperatures (400-500° C.). Yields of pyrolysis oil can exceed 70% w/w. There are several kinds of fast pyrolysis reactors that may be used in the present invention. Particular embodiments comprise a reactor selected from the group consisting of a bubbling fluidized bed, a circulating fluidized beds/transport reactor, a rotating cone pyrolyzer, an ablative pyrolyzer a vacuum pyrolyzer and an Auger reactor. Fluidized bed reactors with either bubbling or circulating media are most commonly used for fast pyrolysis. Auger reactors are also used due to their simplicity and ease of control, but they do not achieve the rapid heat-rates obtained with fluidized bed reactors. Sadaka and Boateng (2009) provide a review of reactor types used for pyrolysis.

During the pyrolysis process, the organic components of biomass (i.e. cellulose, hemicellulose, and lignin) are broken down and depolymerized to form a mixture of vapours and an aersol of micron-sized droplets. Prolonging the reaction time promotes secondary reactions of the aerosols and increases the formation of low molecular weight hydrocarbons (e.g., CH₄, C₂H₆, etc.) and synthesis gas (CO and CO₂ and/or H₂). Rapid cooling and condensing of the mixture forms pyrolysis oil. In a particular embodiment, at least a portion of the pyrolysis gas is passed to the bioreactor as part of the substrate comprising CO.

Pyrolysis oil (approximately represented by C₆H₈O₄) is a complex mixture of oxygenated organic compounds (e.g., acids, alcohols, aldehydes, esters, furans, ketones, sugars, phenols and many multifunctional compounds) and water (typically around 15-30% w/w). On an elemental basis, it is compositionally similar to the parent biomass, hence it is sometimes called “liquid plant matter”.

Biomass-derived pyrolysis oil is rich in carbon and can be refined in ways similar to crude petroleum. Coupled with its ease of transport and storage as compared to solid biomass material, pyrolysis oil can serve as a potential feedstock for the production of fuels and chemicals in petroleum refineries. Pyrolysis oil may be used to produce biofuels including transportation fuel. While the pyrolysis oil may be used in an unprocessed form, post-processing may be desirable to optimise pyrolysis oil for particular applications.

In a particular embodiment, the pyrolysis oil is gasified before being used in the fermentation substrate.

Pyrolysis oil contains lower quantities of trace metals and sulfur making it particularly useful as a low-emission combustion fuel. The recovery of pyrolysis oil from the pyrolysis process and separation from co-products such as char may be performed according to known methods.

The relatively high oxygen content of pyrolysis oil reduces its calorific value relative to most fossil fuels (e.g. about half that of heavy fuel oil). This high oxygen and water content can make them inferior to conventional hydrocarbon fuels in particular contexts. Additionally, phase-separation and polymerization of the liquids and corrosion of containers make storage of these liquids difficult.

Pyrolysis oil upgrading can be used to convert pyrolysis oil to gasoline by mild hydrotreating followed by hydrocracking. Such methods are well known in the art. However, hydrogen production is capital intensive and it is desirable to develop methods that increase hydrogen production and recovery efficiency, especially from low-purity streams. In the absence of hydrogen recovery, such streams end up in fuel gas or sent to flare and the high-value hydrogen component is effectively wasted.

The invention provides a method and system whereby an outlet gas stream comprising H₂ passes from the fermentation bioreactor to a hydrogenation zone which receives pyrolysis oil from a pyrolysis zone. The H₂ contacts the pyrolysis oil and hydrogenates the oil to produce a hydrocarbon product. The hydrocarbon product having between 6 and 20 carbons. In one embodiment the hydrocarbon product is high grade kerosene. In one embodiment the hydrogenation zone is operated unders conditions to produce a jet fuel hydrocarbon product. In one embodiment, the hydrogenation occurs in a steam reformer module.

In a particular embodiment, the pyrolysis oil is contacted with an outlet gas stream comprising hydrogen received from the bioreactor. A substrate comprising CO is provided to the bioreactor. The substrate comprises gases that may have been produced as by-products of the pyrolysis process, or an alternative biomass liquefaction process. In a particular embodiment, the substrate comprising CO also comprises H₂. The fermentation process fixes at least a portion of the CO and optionally CO₂ components of the substrate thus resulting in the outlet gas stream having a higher concentration of hydrogen.

The present invention provides a method and system of using the fermentation reaction as a hydrogen purification apparatus then using the hydrogen to upgrade the pyrolysis oil to superior quality biofuels. These high quality end-products can be produced without the need for costly storage or transport of the pyrolysis oil and without the requirement for high purity hydrogen to be obtained and stored for use in the pyrolysis oil upgrading process. The fermentation effectively acts as a hydrogen membrane allowing H₂ to pass through unconverted and to concentrate the H₂ in the outlet gas stream compared to the substrate provided to the bioreactor.

Where the output stream comprises H₂ and unacceptable levels of impurities or other gas species, further purification may be desirable before pyrolysis oil upgrading. Methods of purification will be known to those of skill in the art, and may include the use of a pressure swing adsorption process. A Pressure Swing Adsorption (PSA) process may be used to recover hydrogen from an impure stream or to increase the purity of hydrogen in the stream. The gas stream comprising H₂ enters a molecular sieve system which adsorbs CO₂, CO, CH₄ N₂ and H₂O at high pressure. Hydrogen is able to pass through the sieve and is collected at approximately 65-90% yield (higher yield being associated with lower final H₂ product purity). Once saturated, the sieve is depressurised then the desorbed gases are swept out using the smallest possible quantity of hydrogen product. The extent of regeneration is a function of pressure, as a greater quantity of adsorbed species is released at lower regeneration pressures. This, in turn, leads to greater hydrogen recovery. Therefore, regeneration pressures of close to atmospheric pressure maximize hydrogen recovery. The vessel is then repressurised with hydrogen ready for the next period as adsorber. Commercial systems will typically have three or four vessels to give a smooth operation. A typical gas stream output from the PSA step would include the following: H₂ (approximately 7-27%), CO₂, CO and CH₄.

### Gasification

In a particular embodiment, a solid and/or liquid feedstock undergoes gasification in a gasification zone adapted to receive solid and/or liquid biomass. At least a portion of the gasified product is passed to the bioreactor as part of the substrate comprising CO. In a particular embodiment, the feedstock is a solid pyrolysis product, for example char, or a liquid pyrolysis product, for example pyrolysis oil.

During gasification, the feedstock undergoes the following processes:
a. Dehydration. Occurs at approximately 100° C. Typically the resulting steam is mixed into the gas flow and may be involved with subsequent chemical reactions, notably the water-gas reaction if the temperature is sufficiently high enough (see step 5);
b. pyrolysis process occurs at around 200-300° C. Volatiles are released and char is produced, resulting in up to 70% weight loss for coal. The process is dependent on the properties of the carbonaceous material and determines the structure and composition of the char, which will then undergo gasification reactions.
c. combustion process occurs as the volatile products and some of the char reacts with oxygen to primarily form carbon dioxide and small amounts of carbon monoxide, which provides heat for the subsequent gasification reactions;
d. gasification occurs as the char reacts with carbon, steam and CO₂ to produce carbon monoxide and hydrogen. In addition, the reversible gas phase water gas shift reaction reaches equilibrium very fast at the temperatures in a gasifier. This balances the concentrations of carbon monoxide, steam, carbon dioxide and hydrogen.

### Char Conversion

Char is a solid charcoal produced by pyrolysis of biomass. Char may be referred to as bio-char when it is used for particular purposes, such as a soil amendment to increase soil fertility, raise agricultural productivity or to improve low grade soils. The use of char can reduce deforestation and has been postulated as a method of mitigating global warming by carbon sequestration.

The quality of char varies depending on the source and production process. When used as a soil amendment, char can improve water quality, reduce soil emissions of greenhouse gases, reduce nutrient leaching, reduce soil acidity, and reduce irrigation and fertilizer requirements.

The invention also provides a fermentation process comprising the use of a substrate comprising CO wherein at least a portion of the substrate is produced by a char conversion process.

The char undergoes conversion in a char conversion module in the presence of CO₂ to form CO. Preferably, the CO₂ for conversion is received in the char conversion module via a gas recycling conduit from the bioreactor, the torrefaction zone, the gasification zone and/or the pyrolysis zone.

### Products

The invention provides fermentation products produced by the methods and systems disclosed herein. In a particular embodiment, the fermentation product is an alcohol or diol. In one embodiment the alcohol is ethanol. In an alternative embodiment the diol is 2,3-butanediol. In certain embodiments the one or more fermentation products is ethanol and 2,3-butanediol. Downstream processing of the fermentation products may produce derivatives such as alkanes or other hydrocarbons.

The invention also provides one or more arene compounds that may be obtained by processing of the pyrolysis oil according to the methods and systems described herein. In particular embodiments, the one or more arene compounds may be combined with alkanes to produce other fuels and compounds, particularly transportation fuels.

It will be appreciated that for growth of the bacteria and the production of products to occur, in addition to the CO-containing substrate gas, a suitable liquid nutrient medium will need to be fed to the bioreactor.

In particular embodiments, the fermentation occurs in an aqueous culture medium. In particular embodiments, the fermentation of the substrate takes place in a bioreactor.

The substrate and media may be fed to the bioreactor in a continuous, batch or batch fed fashion. A nutrient medium will contain vitamins and minerals sufficient to permit growth of the micro-organism used. Anaerobic media suitable for fermentation using CO are known in the art. For example, suitable media are described Biebel (2001). In one embodiment of the invention the media is as described in the Examples section herein after.

Typically, the CO will be added to the fermentation reaction in a gaseous state. However, methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October, 2002) could be used for this purpose. Where a “gas stream” is referred to herein, the term also encompasses other forms of transporting the gaseous components of that stream such as the saturated liquid method described above.

### The Gaseous Substrate

The CO-containing substrate may contain any proportion of CO, such as at least about 20% to about 100% CO by volume, from 40% to 95% CO by volume, from 40% to 60% CO by volume, and from 45% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 2%, may also be appropriate, particularly when H₂ and CO₂ are also present.

The presence of H₂ should not be detrimental to hydrocarbon product formation by fermentation. In particular embodiments, the presence of hydrogen results in an improved overall efficiency of alcohol production. For example, in particular embodiments, the substrate may comprise an approximate 2:1, or 1:1, or 1:2 ratio of H₂:CO. In other embodiments, the CO containing substrate comprises less than about 30% H₂, or less than 27% H₂, or less than 20% H₂, or less than 10% H₂, or lower concentrations of H₂, for example, less than 5%, or less than 4%, or less than 3%, or less than 2%, or less than 1%, or is substantially hydrogen free. In still other embodiments, the CO containing substrate comprises greater than 50% H2, or greater than 60% H2, or greater than 70% H2, or greater than 80% H2, or greater than 90% H2. The substrate may also contain some CO₂ for example, such as about 1% to about 80% CO₂ by volume, or 1% to about 30% CO₂ by volume.

### Fermentation Conditions and Microorganisms

Processes for the production of ethanol and other alcohols from gaseous substrates are known. Exemplary processes include those described for example in WO2007/117157, WO2008/115080, WO2009/022925, WO2009/064200, U.S. Pat. No. 6,340,581, U.S. Pat. No. 6,136,577, U.S. Pat. Nos. 5,593,886, 5,807,722 and 5,821,111, each of which is incorporated herein by reference.

The fermentation should desirably be carried out under appropriate fermentation conditions for the production of desirable fermentation products to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition.

In addition, it is often desirable to increase the CO concentration of a substrate stream (or CO partial pressure in a gaseous substrate) and thus increase the efficiency of fermentation reactions where CO is a substrate. Operating at increased pressures allows a significant increase in the rate of CO transfer from the gas phase to the liquid phase where it can be taken up by the micro-organism as a carbon source for the production of fermentation. This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the invention used. However, in general, it is preferred that the fermentation be performed at pressure higher than ambient pressure. Also, since a given CO-to-product conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

By way of example, the benefits of conducting a gas-to-ethanol fermentation at elevated pressures has been described. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO-containing gaseous substrate is such as to ensure that the concentration of CO in the liquid phase does not become limiting. This is because a consequence of CO-limited conditions may be that one or more product is consumed by the culture.

The composition of gas streams used to feed a fermentation reaction can have a significant impact on the efficiency and/or costs of that reaction. For example, O₂ may reduce the efficiency of an anaerobic fermentation process. Processing of unwanted or unnecessary gases in stages of a fermentation process before or after fermentation can increase the burden on such stages (e.g. where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a microorganism defined herein is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

In a particular embodiment, the microorganism is selected from the group of carboxydotrophic acetogenic bacteria comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kiuvi.

In one particular embodiment, the parental microorganism is selected from the cluster of ethanologenic, acetogenic Clostridia comprising the species C. autoethanogenum, C. ljungdahlii, and C. ragsdalei and related isolates. These include but are not limited to strains C. autoethanogenum JAI-1T (DSM10061) [Abrini J, Naveau H, Nyns E-J: Clostridium autoethanogenum, sp. nov., an anaerobic bacterium that produces ethanol from carbon monoxide. Arch Microbiol 1994, 4: 345-351], C. autoethanogenum LBS1560 (DSM19630) [Simpson S D, Forster R L, Tran P T, Rowe M J, Warner I L: Novel bacteria and methods thereof. International patent publication 2009, WO/2009/064200], C. autoethanogenum LBS1561 (DSM23693), C. ljungdahlii PETCT (DSM13528=ATCC 55383) [Tanner R S, Miller L M, Yang D: Clostridium ljungdahlii sp. nov., an Acetogenic Species in Clostridial rRNA Homology Group I. Int J Syst Bacteriol 1993, 43: 232-236], C. ljungdahlii ERI-2 (ATCC 55380) [Gaddy J L: Clostridium stain which produces acetic acid from waste gases. U.S. Pat. No. 5,593,886, 1997], C. ljungdahlii C-01 (ATCC 55988) [Gaddy J L, Clausen E C, Ko C-W: Microbial process for the preparation of acetic acid as well as solvent for its extraction from the fermentation broth. U.S. Pat. No. 6,368,819, 2002], C. ljungdahlii O-52 (ATCC 55989) [Gaddy J L, Clausen E C, Ko C-W: Microbial process for the preparation of acetic acid as well as solvent for its extraction from the fermentation broth. U.S. Pat. No. 6,368,819, 2002], C. ragsdalei PUT (ATCC BAA-622) [Huhnke R L, Lewis R S, Tanner R S: Isolation and Characterization of novel Clostridial Species. International patent 2008, WO 2008/028055], related isolates such as “C. coskatii” [Zahn et al—Novel ethanologenic species Clostridium coskatii (US Patent Application number US20110229947)] and “Clostridium sp.” (Tyurin et al., 2012, J. Biotech Res. 4: 1-12), or mutated strains such as C. ljungdahlii OTA-1 (Tirado-Acevedo O. Production of Bioethanol from Synthesis Gas Using Clostridium ljungdahlii. PhD thesis, North Carolina State University, 2010). These strains form a subcluster within the Clostridial rRNA cluster I, and their 16S rRNA gene is more than 99% identical with a similar low GC content of around 30%. However, DNA-DNA reassociation and DNA fingerprinting experiments showed that these strains belong to distinct species [Huhnke R L, Lewis R S, Tanner R S: Isolation and Characterization of novel Clostridial Species. International patent 2008, WO 2008/028055]. All species of this cluster have a similar morphology and size (logarithmic growing cells are between 0.5-0.7×3-5 μm), are mesophilic (optimal growth temperature between 30-37° C.) and strictly anaerobe [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Moreover, they all share the same major phylogenetic traits, such as same pH range (pH 4-7.5, with an optimal initial pH of 5.5-6), strong autotrophic growth on CO containing gases with similar growth rates, and a similar metabolic profile with ethanol and acetic acid as main fermentation end product, and small amounts of 2,3-butanediol and lactic acid formed under certain conditions. [Tanner R S, Miller L M, Yang D: Clostridium ljungdahlii sp. nov., an Acetogenic Species in Clostridial rRNA Homology Group I. Int J Syst Bacteriol 1993, 43: 232-236; Abrini J, Naveau H, Nyns E-J: Clostridium autoethanogenum, sp. nov., an anaerobic bacterium that produces ethanol from carbon monoxide. Arch Microbiol 1994, 4: 345-351; Huhnke R L, Lewis R S, Tanner R S: Isolation and Characterization of novel Clostridial Species. International patent 2008, WO 2008/028055]. Indole production was observed with all three species as well. However, the species differentiate in substrate utilization of various sugars (e.g. rhamnose, arabinose), acids (e.g. gluconate, citrate), amino acids (e.g. arginine, histidine), or other substrates (e.g. betaine, butanol). Moreover some of the species were found to be auxotroph to certain vitamins (e.g. thiamine, biotin) while others were not.

In one embodiment the parental microorganism is Clostridium autoethanogenum or Clostridium ljungdahlii. In one particular embodiment, the microorganism is Clostridium autoethanogenum DSM23693. In another particular embodiment, the microorganism is Clostridium ljungdahlii DSM13528 (or ATCC55383).

The fermentation may be carried out in any suitable bioreactor configured for gas/liquid contact wherein the substrate can be contacted with one or more microorganisms, such as a continuous stirred tank reactor (CSTR), an immobilised cell reactor, a gas-lift reactor, a bubble column reactor (BCR), a membrane reactor, such as a Hollow Fibre Membrane Bioreactor (HFMBR) or a trickle bed reactor (TBR), monolith bioreactor or loop reactors. Also, in some embodiments of the invention, the bioreactor may comprise a first, growth reactor in which the micro-organisms are cultured, and a second, fermentation reactor, to which fermentation broth from the growth reactor is fed and in which most of the fermentation product (e.g. ethanol and acetate) is produced.

According to various embodiments of the invention, the carbon source for the fermentation reaction is syngas derived from gasification. The syngas substrate will typically contain a major proportion of CO, such as at least about 15% to about 75% CO by volume, from 20% to 70% CO by volume, from 20% to 65% CO by volume, from 20% to 60% CO by volume, and from 20% to 55% CO by volume. In particular embodiments, the substrate comprises about 25%, or about 30%, or about 35%, or about 40%, or about 45%, or about 50% CO, or about 55% CO, or about 60% CO by volume. Substrates having lower concentrations of CO, such as 6%, may also be appropriate, particularly when H₂ and CO₂ are also present. In particular embodiments, the presence of hydrogen results in an improved overall efficiency of alcohol production. The gaseous substrate may also contain some CO₂ for example, such as about 1% to about 80% CO₂ by volume, or 1% to about 30% CO₂ by volume.

In accordance with particular embodiments of the invention, the CO content and/or the H₂ content of the reformed substrate stream can be enriched prior to passing the stream to the bioreactor. For example, hydrogen can be enriched using technologies well known in the art, such as pressure swing adsorption, cryogenic separation and membrane separation. Similarly, CO can be enriched using technologies well known in the art, such as copper-ammonium scrubbing, cryogenic separation, COSORB™ technology (absorption into cuprous aluminium dichloride in toluene), vacuum swing adsorption and membrane separation. Other methods used in gas separation and enrichment are detailed in PCT/NZ2008/000275, which is fully incorporated herein by reference.

Typically, the carbon monoxide will be added to the fermentation reaction in a gaseous state. However, the methods of the invention are not limited to addition of the substrate in this state. For example, the carbon monoxide can be provided in a liquid. For example, a liquid may be saturated with a carbon monoxide containing gas and that liquid added to the bioreactor. This may be achieved using standard methodology. By way of example a microbubble dispersion generator (Hensirisak et. al. Scale-up of microbubble dispersion generator for aerobic fermentation; Applied Biochemistry and Biotechnology Volume 101, Number 3/October 2002) could be used for this purpose.

It will be appreciated that for growth of the bacteria and CO-to-alcohol fermentation to occur, in addition to the CO-containing substrate gas, a suitable liquid nutrient medium will need to be fed to the bioreactor. A nutrient medium will contain vitamins and minerals sufficient to permit growth of the micro-organism used. Anaerobic media suitable for the fermentation of ethanol using CO as the sole carbon source are known in the art. For example, suitable media are described in U.S. Pat. Nos. 5,173,429 and 5,593,886 and WO 02/08438, WO2007/117157, WO2008/115080, WO2009/022925, WO2009/058028, WO2009/064200, WO2009/064201, WO2009/113878 and WO2009/151342 referred to above. The present invention provides a novel media which has increased efficacy in supporting growth of the micro-organisms and/or alcohol production in the fermentation process. This media will be described in more detail hereinafter.

The fermentation should desirably be carried out under appropriate conditions for the desired fermentation to occur (e.g. CO-to-ethanol). Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. Suitable conditions are described in WO02/08438, WO2007/117157, WO2008/115080, WO2009/022925, WO2009/058028, WO2009/064200, WO2009/064201, WO2009/113878 and WO2009/151342 all of which are incorporated herein by reference.

The optimum reaction conditions will depend partly on the particular micro-organism used. However, in general, it is preferred that the fermentation be performed at pressure higher than ambient pressure. Operating at increased pressures allows a significant increase in the rate of CO transfer from the gas phase to the liquid phase where it can be taken up by the micro-organism as a carbon source for the production of ethanol. This in turn means that the retention time (defined as the liquid volume in the bioreactor divided by the input gas flow rate) can be reduced when bioreactors are maintained at elevated pressure rather than atmospheric pressure.

The benefits of conducting a gas-to-ethanol fermentation at elevated pressures have also been described elsewhere. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO and H2 containing gaseous substrate is such as to ensure that the concentration of CO in the liquid phase does not become limiting. This is because a consequence of CO-limited conditions may be that the ethanol product is consumed by the culture.

Fermentation products such as ethanol, or mixed streams containing more than one fermentation products, may be recovered from the fermentation broth by methods known in the art, such as fractional distillation or evaporation, pervaporation, gas stripping and extractive fermentation, including for example, liquid-liquid extraction. Products may also diffuse or secrete into media, from which they can extracted by phase separation.

In certain preferred embodiments of the invention, one or more products are recovered from the fermentation broth by continuously removing a portion of the broth from the bioreactor, separating microbial cells from the broth (conveniently by filtration), and recovering one or more products from the broth. Alcohols may conveniently be recovered for example by distillation. Acetone may be recovered for example by distillation. Any acids produced may be recovered for example by adsorption on activated charcoal. The separated microbial cells are preferably returned to the fermentation bioreactor. The cell free permeate remaining after any alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor

### Product Recovery

Then products of the fermentation reaction can be recovered using known methods. Exemplary methods include those described in WO2007/117157, WO2008/115080, WO2009/022925, U.S. Pat. Nos. 6,340,581, 6,136,577, 5,593,886, 5,807,722 and 5,821,111. However, briefly and by way of example only ethanol may be recovered from the fermentation broth by methods such as fractional distillation or evaporation, and extractive fermentation.

Distillation of ethanol from a fermentation broth yields an azeotropic mixture of ethanol and water (i.e., 95% ethanol and 5% water). Anhydrous ethanol can subsequently be obtained through the use of molecular sieve ethanol dehydration technology, which is also well known in the art.

Extractive fermentation procedures involve the use of a water-miscible solvent that presents a low toxicity risk to the fermentation organism, to recover the ethanol from the dilute fermentation broth. For example, oleyl alcohol is a solvent that may be used in this type of extraction process. Oleyl alcohol is continuously introduced into a fermenter, whereupon this solvent rises forming a layer at the top of the fermenter which is continuously extracted and fed through a centrifuge. Water and cells are then readily separated from the oleyl alcohol and returned to the fermenter while the ethanol-laden solvent is fed into a flash vaporization unit. Most of the ethanol is vaporized and condensed while the oleyl alcohol is non volatile and is recovered for re-use in the fermentation.

Acetate, which is produced as by-product in the fermentation reaction, may also be recovered from the fermentation broth using methods known in the art. For example, an adsorption system involving an activated charcoal filter may be used. In this case, it is preferred that microbial cells are first removed from the fermentation broth using a suitable separation unit. Numerous filtration-based methods of generating a cell free fermentation broth for product recovery are known in the art. The cell free ethanol—and acetate—containing permeate is then passed through a column containing activated charcoal to adsorb the acetate. Acetate in the acid form (acetic acid) rather than the salt (acetate) form is more readily adsorbed by activated charcoal. It is therefore preferred that the pH of the fermentation broth is reduced to less than about 3 before it is passed through the activated charcoal column, to convert the majority of the acetate to the acetic acid form.

Acetic acid adsorbed to the activated charcoal may be recovered by elution using methods known in the art. For example, ethanol may be used to elute the bound acetate. In certain embodiments, ethanol produced by the fermentation process itself may be used to elute the acetate. Because the boiling point of ethanol is 78.8° C. and that of acetic acid is 107° C., ethanol and acetate can readily be separated from each other using a volatility-based method such as distillation.

Other methods for recovering acetate from a fermentation broth are also known in the art and may be used in the processes of the present invention. For example, U.S. Pat. Nos. 6,368,819 and 6,753,170 describe a solvent and cosolvent system that can be used for extraction of acetic acid from fermentation broths. As with the example of the oleyl alcohol-based system described for the extractive fermentation of ethanol, the systems described in U.S. Pat. Nos. 6,368,819 and 6,753,170 describe a water immiscible solvent/co-solvent that can be mixed with the fermentation broth in either the presence or absence of the fermented micro-organisms in order to extract the acetic acid product. The solvent/co-solvent containing the acetic acid product is then separated from the broth by distillation. A second distillation step may then be used to purify the acetic acid from the solvent/co-solvent system.

The products of the fermentation reaction (for example ethanol and acetate) may be recovered from the fermentation broth by continuously removing a portion of the broth from the fermentation bioreactor, separating microbial cells from the broth (conveniently by filtration), and recovering one or more product from the broth simultaneously or sequentially. In the case of ethanol it may be conveniently recovered by distillation, and acetate may be recovered by adsorption on activated charcoal, using the methods described above. The separated microbial cells are preferably returned to the fermentation bioreactor. The cell free permeate remaining after the ethanol and acetate have been removed is also preferably returned to the fermentation bioreactor. Additional nutrients (such as B vitamins) may be added to the cell free permeate to replenish the nutrient medium before it is returned to the bioreactor. Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

### General

Embodiments of the invention are described by way of example. However, it should be appreciated that particular steps or stages necessary in one embodiment may not be necessary in another. Conversely, steps or stages included in the description of a particular embodiment can be optionally advantageously utilised in embodiments where they are not specifically mentioned.

While the invention is broadly described with reference to any type of stream that may be moved through or around the system(s) by any known transfer means, in certain embodiments, the biogas and reformed and/or blended substrate streams are gaseous. Those skilled in the art will appreciate that particular stages may be coupled by suitable conduit means or the like, configurable to receive or pass streams throughout a system. A pump or compressor may be provided to facilitate delivery of the streams to particular stages. Furthermore, a compressor can be used to increase the pressure of gas provided to one or more stages, for example the bioreactor. As discussed hereinabove, the pressure of gases within a bioreactor can affect the efficiency of the fermentation reaction performed therein. Thus, the pressure can be adjusted to improve the efficiency of the fermentation. Suitable pressures for common reactions are known in the art.

In addition, the systems or processes of the invention may optionally include means for regulating and/or controlling other parameters to improve overall efficiency of the process. For example particular embodiments may include determining means to monitor the composition of substrate and/or exhaust stream(s). In addition, particular embodiments may include a means for controlling the delivery of substrate stream(s) to particular stages or elements within a particular system if the determining means determines the stream has a composition suitable for a particular stage. For example, in instances where a gaseous substrate stream contains low levels of CO or high levels of O₂ that may be detrimental to a fermentation reaction, the substrate stream may be diverted away from the bioreactor. In particular embodiments of the invention, the system includes means for monitoring and controlling the destination of a substrate stream and/or the flow rate, such that a stream with a desired or suitable composition can be delivered to a particular stage.

In addition, it may be necessary to heat or cool particular system components or substrate stream(s) prior to or during one or more stages in the process. In such instances, known heating or cooling means may be used.

Various embodiments of the systems of the invention are described in the accompanying Figures.

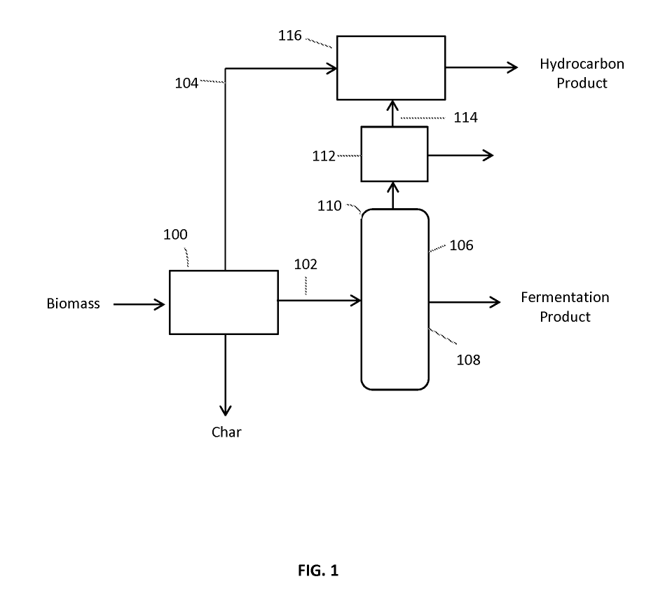

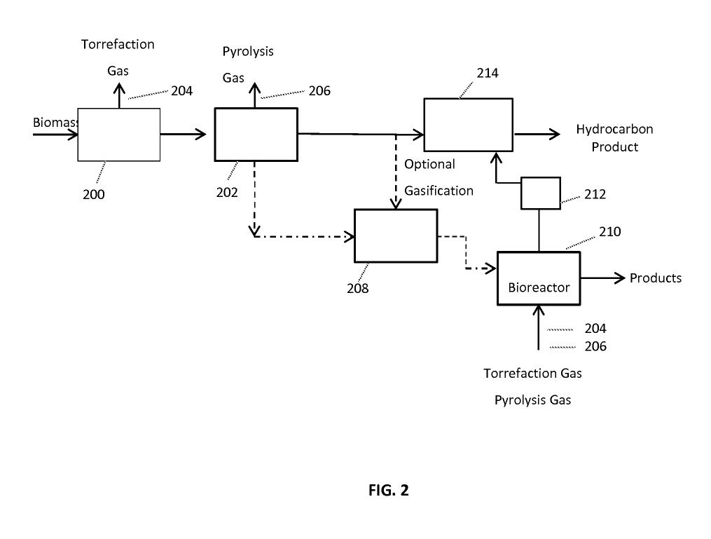

Alternative embodiments of the invention are described in FIGS. 1 and 2. As shown in FIG. 1, one embodiment of the invention provides a system and method for the production of one or more products, the system including;
a. a pyrolysis zone 100 wherein a biomass feedstock is reacted under pyrolysis conditions to produce a gaseous substrate and at least one pyrolysis product, selected from the group consisiting of pyrolysis and char.
b. a bioreactor 106 adapted to receive the gaseous substrate from the pyrolysis zone via a conduit 102. The bioreactor 106 contains a culture of at least one carboxydotrophic acetogenic microorganism in a liquid nutrient medium broth. The bioreactor is opereated under fermentation conditions to produce at least one fermentation product and an exit gas stream comprising H2. The at least one fermentation product is removed from the reactor via a product conduit 108.
c. the exit stream is passed from the bioreactor via a gas conduit to a gas separation zone 112, where a hydrogen portion of the exit gas stream is separated and passed via a conduit 114 to a hydrogenation zone 116.
d. The hydrogenation zone 116 is adapted to receive a hydrogen stream from the gas separation zone and the pyrolysis oil from the pyrolysis zone via a conduit 104. The pyrolysis oil and the hydrogen are reacted under hydrogenation conditions to produce a hydrocarbon product having between 6 and 20 carbons.

FIG. 2 shows an alternative embodiment for the production of one or more products by fermentation of a gasesous substrate produced by liquefaction of biomass. According to one embodiment of the invention, the biomass feedstock is passed to a Torrefaction zone 200 wherein the torrefaction zone operates under conditions to produce a torrefied biomass and a torrefaction gas stream 204. The torrefied biomass is passed to a pyrolysis zone 202. The torrified biomass is reacted under pyrolysis conditions to produce a pyrolysis gas stream 206, pyrolysis oil and char. At least a portion of the torrefecation gas stream 204 and the pyrolysis gas stream 206 are passed to a bioreactor 210.

At least a portion of the pyrolysis oil and/or char can optionally be passed to a gasification zone 208 where they are gasified to produce a gasification substrate comprising CO. The gasification substrate can be passed to the bioreactor 210. The bioreactor 210 contains a culture of at least one carboxydotrophic acetogenic microorganism in a liquid nutrient medium broth. The bioreactor is opereated under fermentation conditions to produce at least one fermentation product and an exit gas stream comprising H2. The exit stream is passed to a gas separation zone 212 where hydrogen is separated from the gas stream to produce an enriched hydrogen stream. The enriched hydrogen stream is passed to a hydrogenation zone 214. The hydrogenation zone 214 is adapted to receive the pyrolysis oil from the pyrolysis zone 206 and the enriched hydrogen stream. The pyrolysis oil and hydrogen stream are reacted under hydrogenation coniditons to produce a hydrocarbon having between 6 and 20 carbons.

The invention has been described herein, with reference to certain preferred embodiments, in order to enable the reader to practice the invention without undue experimentation. However, a person having ordinary skill in the art will readily recognise that many of the components and parameters may be varied or modified to a certain extent or substituted for known equivalents without departing from the scope of the invention. It should be appreciated that such modifications and equivalents are herein incorporated as if individually set forth. Titles, headings, or the like are provided to enhance the reader's comprehension of this document, and should not be read as limiting the scope of the present invention.

The entire disclosures of all applications, patents and publications, cited above and below, if any, are hereby incorporated by reference. However, the reference to any applications, patents and publications in this specification is not, and should not be taken as an acknowledgment or any form of suggestion that they constitute valid prior art or form part of the common general knowledge in any country in the world.

Throughout this specification and any claims which follow, unless the context requires otherwise, the words “comprise”, “comprising” and the like, are to be construed in an inclusive sense as opposed to an exclusive sense, that is to say, in the sense of “including, but not limited to”.

What we claim is:

1. A method for producing at least one product from a gaseous substrate, the method comprising;
a. Converting at least a portion of a biomass feedstock to a gaseous substrate comprising CO by a biomass liquefaction process selected from pyrolysis or torrefaction the process carried out in a liquefaction zone;
b. Passing at least a portion of the gaseous substrate to a bioreactor comprising a culture of at least one carboxydotrophic acetogenic microorganism, and anaerobically fermenting at least a portion of the gaseous substrate to produce at least one fermentation product and a waste stream comprising a second biomass;
c. separating at least a portion of the second biomass from the waste stream; and
d. Passing a portion of the second biomass to the liquefaction zone.

2. The method of claim 1, wherein the gaseous substrate further comprises CO₂ and H₂.

3. The method of claim 2 wherein the biomass liquefaction process produces at least one non-gaseous product, and said non-gaseous product is gasified to produce a syngas stream comprising CO.

4. The method of claim 3 wherein at least a portion of the syngas stream is passed to the bioreactor.

5. The method of claim 1 wherein the at least one fermentation product is selected from the group consisting of ethanol, acetic acid and 2,3-butanediol.

6. A method for producing at least one product from a gaseous substrate, the method comprising;
a. Passing a biomass feedstock to a pyrolysis zone, operated at conditions to produce a gaseous substrate comprising CO, CO₂ and H₂, and at least one pyrolysis product, selected from the group consisting of pyrolysis oil and char;
b. Passing at least a portion of the gaseous substrate to a bioreactor comprising a culture of at least one carboxydotrophic acetogenic microorganism, and anaerobically fermenting at least a portion of the gaseous substrate to produce at least one fermentation product, a waste stream comprising a second biomass and an exit gas stream comprising hydrogen;
c. separating at least a portion of the second biomass from the waste stream; and
d. Passing a portion of the second biomass to the pyrolysis zone.

7. The method of claim 6 wherein the exit gas stream is passed to a separation zone operated at conditions to provide a hydrogen rich stream.

8. The method of claim 7 where the pyrolysis product is pyrolysis oil and the hydrogen rich stream and the pyrolysis oil are passed to a hydrogenation zone operated at conditions to produce a hydrogenated product.

9. The method of claim 8 wherein the hydrogenated product is a hydrocarbon having from 6 to 20 carbons.

10. The method of claim 6 further comprising passing the gaseous substrate from the pyrolysis zone to a separation zone operated at conditions to provide a CO₂ rich stream and an enriched CO and H₂ gaseous substrate and passing the enriched gaseous substrate to the bioreactor.

11. The method of claim 10 where the pyrolysis product is char and the char and the CO₂ rich stream are passed to a reaction zone to produce a second substrate stream comprising CO, and passing the second substrate stream to the bioreactor.

12. The method of claim 6, further comprising prior to passing the biomass feedstock to the pyrolysis zone, the biomass feedstock is first passed to a torrefaction zone.

13. A method for producing at least one product from a gaseous substrate, the method comprising;
a. Passing a biomass feedstock to a torrefaction zone to produce a torrefied biomass;
b. Passing the torrefied biomass to a pyrolysis zone to produce a gaseous substrate comprising CO, pyrolysis oil and char;
c. Passing at least a portion of the gaseous substrate to a bioreactor comprising a culture of at least one carboxydotrophic acetogenic microorganism, and anaerobically fermenting at least a portion of the gaseous substrate to produce at least one fermentation product, a waste stream comprising a second biomass and an exit gas stream comprising hydrogen;
d. separating at least a portion of the second biomass from the waste stream;
e. passing the exit gas stream to a separation zone operated at conditions to provide a hydrogen rich stream;
f. Passing a portion of the second biomass to the torrefaction zone; and
g. Passing the pyrolysis oil and the hydrogen rich stream to a hydrogenation zone operated at conditions to produce a hydrogenated product.

14. The method of claim 13 wherein the torrefaction process produces a gaseous by-product stream comprising CO, and at least a portion of the gaseous by-product stream is passed to the bioreactor.

15. The method of claim 13 wherein the char is passed to a gasification zone and gasified to produce a second gaseous substrate comprising CO, and passing the second gaseous substrate to the bioreactor.

16. The method of claim 13 wherein the hydrogenated product is a hydrocarbon having from 6 to 20 carbons.

17. The method of claim 13 wherein the carboxydotrophic acetogenic microorganism is selected from the group consisting of Clostridium autoethanogenum, Clostridium ljundahlii, Clostridium ragsdalei and Clostridium coskatii.
